(19) United States
(12) Patent Application Publication
(10) Pub. No.: US 2010/0305537 A1
(43) Pub. Date: Dec. 2, 2010

(54) Better Fitting Diaper Or Pant With Absorbent Particulate Polymer Material And Preformed Crotch

(76) Inventor: Gregory Ashton, (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/476,269

(22) Filed: Jun. 2, 2009

(51) Int. Cl. A61F 13/15; B32B 37/00

(52) U.S. Cl. 604/374; 156/60

(57) ABSTRACT

A disposable absorbent article includes an absorbent core that is substantially cellulose free and a chassis that can include a topsheet and a backsheet, with the absorbent core located between the topsheet and the backsheet. The chassis may include an absorbent core which includes an absorbent particulate polymer material, the absorbent core being located between the topsheet and the backsheet, and wherein the absorbent core includes at least one strip extending through a portion of a crotch region of the article. The article can also include at least one longitudinally oriented crease passing through a portion of the crotch region, wherein a concave shape is formed within the article.

### FIELD OF THE INVENTION

The invention generally relates to an absorbent article, and more particularly to a disposable absorbent garment with absorbent particulate polymer material and preformed crotch.

### BACKGROUND OF THE INVENTION

Absorbent articles, such as disposable diapers, training pants, and adult incontinence undergarments, absorb and contain body exudates. They also are intended to prevent body exudates from soiling, wetting, or otherwise contaminating clothing or other articles, such as bedding, that come in contact with the wearer. A disposable absorbent article, such as a disposable diaper, may be worn for several hours in a dry state or in a urine loaded state. Accordingly, efforts have been made toward improving the fit and comfort of the absorbent article to the wearer, both when the article is dry and when the article is fully or partially loaded with liquid exudate, while maintaining or enhancing the absorbing and containing functions of the article.

Some absorbent articles, like diapers, contain an absorbent polymer material (also known as super absorbent polymer), such as an absorbent particulate polymer material. Absorbent particulate polymer material absorbs liquid and swells and may be more effective when disposed in an absorbent article in a certain pattern or arrangement intended for optimal absorbency, fit, and/or comfort. Thus, it may be desirable for absorbent particulate polymer material to remain in its intended location in an absorbent article and absorbent particulate polymer material, therefore, is desirably immobilized in the absorbent article such that the absorbent particulate polymer material remains immobilized when the absorbent article is dry and when it is wet.

In addition to being absorbent, absorbent articles, such as diapers, desirably may be thin and flexible, for ease and comfort in use and for more convenient and neat packaging and storage. Absorbent articles, which may often be used in large quantities, may also desirably be inexpensive. Some technologies of immobilizing absorbent particulate polymer material in an absorbent article add bulk to the absorbent article and thereby increase thickness, reduce flexibility, and/or increase cost of the absorbent article. Other technologies for immobilizing absorbent particulate polymer material in an absorbent article may not be as effective in maintaining immobilization when the absorbent article is in a wet state as when in a dry state. Accordingly, there remains a need for a thin, flexible, and/or inexpensive absorbent article containing absorbent particulate polymer material with enhanced immobilization of the absorbent particulate polymer material in the article in dry and wet states. It would be furthermore desirable to meet this need without reducing, or even while enhancing, effective retention of the free liquid exudate before and during absorption by the absorbent core.

### SUMMARY OF THE INVENTION

Embodiments of the invention can address one or more technical problems described above and can provide a disposable absorbent garment with absorbent particulate polymer material and preformed crotch. In one embodiment, a disposable absorbent article is provided. The article can include an absorbent core with a chassis that can include a topsheet and a backsheet, with the absorbent core located between the topsheet and the backsheet. The chassis may include an absorbent core which includes an absorbent particulate polymer material, the absorbent core being located between the topsheet and the backsheet, and wherein the absorbent core includes at least one strip extending through a portion of a crotch region of the article. The article can also include at least one longitudinally oriented crease passing through a portion of the crotch region, wherein a concave shape is formed within the article.

Methods for making the article are also provided.

Other features and advantages of the invention may be apparent from reading the following detailed description, drawings, and claims.

### BRIEF DESCRIPTION OF THE DRAWINGS

FIG. 1 is a plan view of a diaper in accordance with an embodiment of the invention.

FIG. 2 is a cross sectional view of the diaper shown in FIG. 1 taken along the sectional line 2-2 of FIG. 1.

FIG. 3 is an overhead view of an absorbent core layer in accordance with an embodiment of the invention.

FIG. 4 is a cross sectional view of an absorbent core layer shown in FIG. 3 taken along the sectional line 4-4 of FIG. 3.

FIG. 5 is an overhead view of another absorbent core layer in accordance with an embodiment of the invention.

FIG. 6 is a schematic illustration of a process for making an absorbent core in accordance with an embodiment of the invention.

FIG. 7 is a partial sectional view of an apparatus for making an absorbent core in accordance with an embodiment of the invention.

FIG. 8 is a perspective view of the printing roll illustrated in FIG. 7.

FIG. 9 is a partial sectional view of the printing roll illustrated in FIG. 8 showing an absorbent particulate polymer material reservoir.

FIG. 10 is a perspective view of the supporting roll illustrated in FIG. 8.

FIG. 11 is a schematic illustration of a process for making a diaper with an absorbent core in accordance with an embodiment of the invention

### DETAILED DESCRIPTION OF EMBODIMENTS OF THE INVENTION

As summarized above, embodiments of the invention may encompass an absorbent article, such as a diaper, having an absorbent core and a preformed crotch. The combination of an absorbent core and preformed crotch advantageously may provide the disposable absorbent article with improved compressibility, flexibility, and conformity to a wearer's body for greater comfort without increasing the likelihood of leakage from the disposable absorbent article.

Section A below describes terms for assisting the reader in understanding features of the invention, but not introducing limitations in the terms inconsistent with the context with which they are used in the specification. Section B is a detailed description for absorbent articles made in accordance with embodiments of this invention. Section C describes methods of manufacturing absorbent cores and absorbent articles in accordance with embodiments of this invention.

### A. Terms

“Absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” means a structure typically disposed between a topsheet and backsheet of an absorbent article for absorbing and containing liquid received by the absorbent article and may comprise one or more substrates, absorbent polymer material disposed on the one or more substrates, and a thermoplastic composition on the absorbent particulate polymer material and at least a portion of the one or more substrates for immobilizing the absorbent particulate polymer material on the one or more substrates. In a multilayer absorbent core, the absorbent core may also include a cover layer. The one or more substrates and the cover layer may comprise a nonwoven. Further, the absorbent core is substantially cellulose free. The absorbent core does not include an acquisition system, a topsheet, or a backsheet of the absorbent article. In a certain embodiment, the absorbent core would consist essentially of the one or more substrates, the absorbent polymer material, the thermoplastic composition, and optionally the cover layer.

“Absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01).

“Absorbent particulate polymer material” is used herein to refer to an absorbent polymer material which is in particulate form so as to be flowable in the dry state.

“Airfelt” is used herein to refer to comminuted wood pulp, which is a form of cellulosic fiber.

“Compressed” or “compression” is used to describe moving fibers or components of a material relatively closer together.

“Comprise,” “comprising,” and “comprises” are open ended terms, each specifies the presence of what follows, e.g., a component, but does not preclude the presence of other features, e.g., elements, steps, components known in the art, or disclosed herein.

“Concave” is used herein to describe a curved surface which bends, narrows, or otherwise slopes slightly inward. Examples of suitable concave shapes can be, but are not limited to, a V-shape, a U-shape, and any other similar shape that bends, narrows, or slopes slightly inward.

“Consisting essentially of” is used herein to limit the scope of subject matter, such as that in a claim, to the specified materials or steps and those that do not materially affect the basic and novel characteristics of the subject matter.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Elastic”, “elastically extensible”, and “elasticized” refer herein to the property of a material and/or an element of a diaper or other disposable absorbent article whereby the material and/or the element can be elongated to at least 150% of its original unstretched length without rupture or catastrophic failure upon the application of tensioning force and will substantially return to its original length or near its original length after the tension is released.

“Fiber” and “filament” are used interchangeably.

A “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yam). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999.

“Substantially cellulose free” is used herein to describe an article, such as an absorbent core, that contains less than 10% by weight cellulosic fibers, less than 5% cellulosic fibers, less than 1% cellulosic fibers, no cellulosic fibers, or no more than an immaterial amount of cellulosic fibers. An immaterial amount of cellulosic material would not materially affect the thinness, flexibility, or absorbency of an absorbent core.

“Thermoplastic adhesive material” as used herein is understood to comprise a polymer composition from which fibers are formed and applied to the superabsorbent material with the intent to immobilize the superabsorbent material in both the dry and wet state. The thermoplastic adhesive material of the present invention forms a fibrous network over the superabsorbent material.

“Thickness” and “caliper” are used herein interchangeably. B. Absorbent Articles

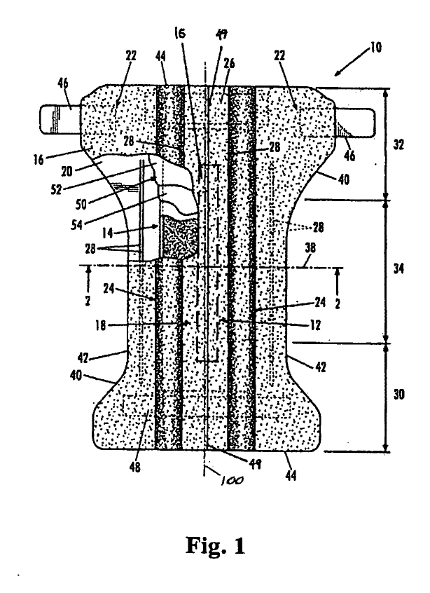

FIG. 1 is a plan view of a diaper 10 according to a certain embodiment of the invention. The diaper 10 is shown in its flat out, uncontracted state (i.e., without elastic induced contraction) and portions of the diaper 10 are cut away to more clearly show the underlying structure of the diaper 10. A portion of the diaper 10 that contacts a wearer is facing the viewer in FIG. 1. The diaper 10 generally may comprise a chassis 12 and an absorbent core 14 disposed in the chassis. The absorbent core 14 may include one or more strips 15. The strip 15 may be a discontinuity in a homogeneous member produced by cutting, adhering, shearing, leaving a gap, perforation, compressing, or any other method of creating a discontinuity. In a certain embodiment, the absorbent core 14 may include one or more compressed strips 15. The compressed strip 15 can be provided by way of mechanical or pressurized compression of a selected portion of the absorbent core 14.

As shown in FIGS. 3 and 4, the absorbent core 14 can include at least one compressed strip 15 generally positioned in a central portion of the core 14. The compressed strip 15 shown is generally aligned parallel with and along the transverse axis 100 of the absorbent core 14. As shown in FIG. 4, which is a sectional view taken along sectional line 4-4 in FIG. 3, the compressed strip 15 is a slightly compressed portion of the absorbent core 14 relative to the other, non-compressed material comprising the absorbent core 14. In other certain embodiments, a compressed strip can be offset from the transverse axis 100 and/or may be segmented into multiple parts. The compressed strip 15 shown can be made, for example, by nip rolls. In certain other embodiments, for example in FIG. 5, an absorbent core 14 can include more than one compressed strip, for instance, two compressed strips 102 and 104 generally aligned parallel with and along the transverse axis 100 of the absorbent core 14.

The chassis 12 of the diaper 10 in FIG. 1 may comprise the main body of the diaper 10. The chassis 12 may comprise an outer covering 16 including a topsheet 18, which may be liquid pervious, and/or a backsheet 20, which may be liquid impervious. The absorbent core 14 may be encased between the topsheet 18 and the backsheet 20. The chassis 12 may also include side panels 22, elasticized leg cuffs 24, and an elastic waist feature 26.

The leg cuffs 24 and the elastic waist feature 26 may each typically comprise elastic members 28. One end portion of the diaper 10 may be configured as a first waist region 30 of the diaper 10. An opposite end portion of the diaper 10 may be configured as a second waist region 32 of the diaper 10. An intermediate portion of the diaper 10 may be configured as a crotch region 34, which extends longitudinally between the first and second waist regions 30 and 32. The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 is depicted in FIG. 1 with its longitudinal axis 36 and its transverse axis 38. The periphery 40 of the diaper 10 is defined by the outer edges of the diaper 10 in which the longitudinal edges 42 run generally parallel to the longitudinal axis 36 of the diaper 10 and the end edges 44 run between the longitudinal edges 42 generally parallel to the transverse axis 38 of the diaper 10. The chassis 12 may also comprise a fastening system, which may include at least one fastening member 46 and at least one stored landing zone 48.

In certain embodiments, an absorbent core 14 can include a compressed strip 15 oriented substantially parallel with the longitudinal axis 36 of the absorbent core 14. The length of the compressed strip 15 can, for example, generally extend within or through the crotch region 34 of the absorbent core 14, and substantially between end edges 44 of the diaper 10. In certain embodiments, for example shown in FIG. 5, multiple compressed strips 102, 104 can be oriented substantially parallel with the longitudinal axis 36 of the absorbent core 14. The lengths of multiple compressed strips 102, 104 can, for example, generally extend within or through the central region of the absorbent core 14, and substantially between end edges 44 of the diaper 10. Each of the multiple compressed strips 102, 104 could have similar or different lengths. In any embodiment, a compressed strip 15 can be provided in the absorbent core 14 by way of mechanical or pressurized compression of a selected portion of the absorbent core 14.

The diaper 10 shown in FIG. 1 can also include one or more creases 49 oriented substantially parallel with the longitudinal axis 36 and extending substantially between the end edges 44. In certain embodiments, one or more creases can extend in directions generally along the longitudinal axis 36, or may extend partially between the end edges 44. In certain other embodiments, one or more creases can include multiple creases oriented substantially parallel with the longitudinal axis 36 and extending substantially between the end edges 44. In other certain embodiments, one or more creases can be slightly offset from the transverse axis 100 and/or each crease may be segmented into multiple parts. As shown in the embodiment of FIG. 1, the crease 49 can extend substantially through the compressed strip 15. In certain embodiments, a crease 49 can extend substantially through multiple compressed strips, or in some certain embodiments, a crease may extend adjacent to multiple compressed strips.

For certain embodiments when the absorbent core 14 may be relatively incompressible, such as an airfelt free core, i.e. with little or no wood pulp or cellulosic material, a crease 49 can include an auxiliary adhesive which is not illustrated in the figures. The auxiliary adhesive may be deposited in the crease 49 when the diaper 10 is folded or compressed along the crease 49. The auxiliary glue may aid in maintaining the relative shape of the crease 49 in the diaper 10. An auxiliary adhesive can include, but is not limited to, sprayable hot melt adhesives, such as H.B. Fuller Co. (St. Paul, Minn.) Product No. HL-1620-B.

The diaper 10 may also include such other features as are known in the art including front and rear ear panels, waist cap features, elastics and the like to provide better fit, containment and aesthetic characteristics. Such additional features are well known in the art and are e.g., described in U.S. Pat. No. 3,860,003 and U.S. Pat. No. 5,151,092.

In order to keep the diaper 10 in place about the wearer, at least a portion of the first waist region 30 may be attached by the fastening member 46 to at least a portion of the second waist region 32 to form leg opening(s) and an article waist. When fastened, the fastening system carries a tensile load around the article waist. The fastening system may allow an article user to hold one element of the fastening system, such as the fastening member 46, and connect the first waist region 30 to the second waist region 32 in at least two places. This may be achieved through manipulation of bond strengths between the fastening device elements.

According to certain embodiments, the diaper 10 may be provided with a re-closable fastening system or may alternatively be provided in the form of a pant-type diaper. When the absorbent article is a diaper, it may comprise a re-closable fastening system joined to the chassis for securing the diaper to a wearer. When the absorbent article is a pant-type diaper, the article may comprise at least two side panels joined to the chassis and to each other to form a pant. The fastening system and any component thereof may include any material suitable for such a use, including but not limited to plastics, films, foams, nonwoven, woven, paper, laminates, fiber reinforced plastics and the like, or combinations thereof. In certain embodiments, the materials making up the fastening device may be flexible. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the diaper 10 with other features added to form the composite diaper structure. While the topsheet 18, the backsheet 20, and the absorbent core 14 may be assembled in a variety of well-known configurations, exemplary diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

The topsheet 18 in FIG. 1 may be fully or partially elasticized or may be foreshortened to provide a void space between the topsheet 18 and the absorbent core 14. Exemplary structures including elasticized or foreshortened topsheets are described in more detail in U.S. Pat. No. 5,037,416 entitled “Disposable Absorbent Article Having Elastically Extensible Topsheet” issued to Allen et al. on Aug. 6, 1991; and U.S. Pat. No. 5,269,775 entitled “Trisection Topsheets for Disposable Absorbent Articles and Disposable Absorbent Articles Having Such Trisection Topsheets” issued to Freeland et al. on Dec. 14, 1993.

The backsheet 26 may be joined with the topsheet 18. The backsheet 20 may prevent the exudates absorbed by the absorbent core 14 and contained within the diaper 10 from soiling other external articles that may contact the diaper 10, such as bed sheets and undergarments. In certain embodiments, the backsheet 26 may be substantially impervious to liquids (e.g., urine) and comprise a laminate of a nonwoven and a thin plastic film such as a thermoplastic film having a thickness of about 0.012 mm (0.5 mil) to about 0.051 mm (2.0 mils). Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper 10 while still preventing exudates from passing through the backsheet 10. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E.I. DuPont. Other breathable backsheets including nonwoven webs and apertured formed films are described in U.S. Pat. No. 5,571,096 issued to Dobrin et al. on Nov. 5, 1996.

In certain embodiments, the backsheet of the present invention may have a water vapor transmission rate (WVTR) of greater than about 2000 g/24 h/m², greater than about 3000 g/24 h/m², greater than about 5000 g/24 h/m², greater than about 6000 g/24 h/m², greater than about 7000 g/24 h/m², greater than about 8000 g/24 h/m², greater than about 9000 g/24 h/m², greater than about 10000 g/24 h/m², greater than about 11000 g/24 h/m², greater than about 12000 g/24 h/m², greater than about 15000 g/24 h/m², measured according to WSP 70.5 (08) at 37.8° C. and 60% Relative Humidity.

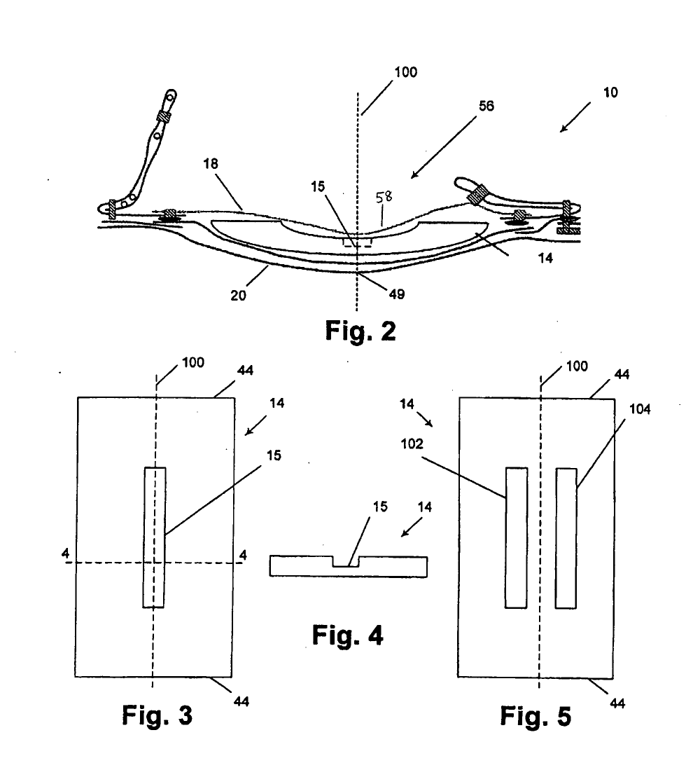

FIG. 2 shows a cross section of FIG. 1 taken along the sectional line 2-2 of FIG. 1. Starting from the wearer facing side 56, the diaper 10 may comprise the topsheet 18, the components of the absorbent core 14, and the backsheet 20. As shown in FIGS. 1 and 2, the diaper 10 can include at least one compressed strip 15 aligned with a crease 49, which together can cooperate to facilitate formation of a concave shape 58 in the diaper 10. The concave shape is also known as a “preformed concave shape” or “preformed shaped” since the concave shape is formed in the diaper 10 prior to use of the diaper 10 by a wearer. The concave shape 58 can have the shape of a slight trough oriented generally in the crotch region 34 of the diaper 10. In certain embodiments, a diaper with a preformed concave shape can relatively increase bucket volume over conventional diapers. This aspect may be attributed to the concave shape 58 which can generally receive fluids or liquid exudates within the cavity of the concave shape 58. Furthermore, a diaper with a concave shape can have improved fit between a wearer's legs. This aspect may be attributed to the concave shape 58 which generally conforms with the shape of a wearer's body, and thus reduces the relative amount of the diaper 10 which may not conform with the wearer's body.

In certain embodiments, a concave shape can be a concave V-shape. In other certain embodiments, a concave shape can be a concave U-shape. In certain other embodiments, a concave shape can be a surface that bends, narrows, or slopes slightly inward.

According to a certain embodiment, diaper 10 may also comprise an acquisition system 50 disposed between the liquid permeable topsheet 18 and a wearer facing side of the absorbent core 14. The acquisition system shown in FIG. 1 can include an upper acquisition layer 52 and a lower acquisition layer 54. The acquisition system 50 may, for example, comprise chemically cross-linked cellulosic fibers. Such cross-linked cellulosic fibers may have desirable absorbency properties. Exemplary chemically cross-linked cellulosic fibers are disclosed in U.S. Pat. No. 5,137,537. In certain embodiments, the chemically cross-linked cellulosic fibers are cross-linked with between about 0.5 mole % and about 10.0 mole % of a C₂ to C₉ polycarboxylic cross-linking agent or between about 1.5 mole % and about 6.0 mole % of a C₂ to C₉ polycarboxylic cross-linking agent based on glucose unit. Citric acid is an exemplary cross-linking agent. In other embodiments, polyacrylic acids may be used. Further, according to certain embodiments, the cross-linked cellulosic fibers have a water retention value of about 25 to about 60, or about 28 to about 50, or about 30 to about 45. A method for determining water retention value is disclosed in U.S. Pat. No. 5,137,537. According to certain embodiments, the cross-linked cellulosic fibers may be crimped, twisted, or curled, or a combination thereof including crimped, twisted, and curled.

In a certain embodiment, the acquisition system 50 may comprise a non-woven, which may be hydrophilic. Further, according to a certain embodiment, the acquisition system 50 may comprise the chemically cross-linked cellulosic fibers, which may or may not form part of a nonwoven material. According to another embodiment, the acquisition system 50 may comprise a combination of nonwovens, at least one nonwoven without the cross-linked cellulosic fibers and at least one nonwoven with chemically cross-linked cellulosic fibers. Further, according to an embodiment, the acquisition system 50 may comprise the chemically cross-linked cellulosic fibers mixed with other fibers such as natural or synthetic polymeric fibers. According to other embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof.

According to a certain embodiment, the acquisition system 50 desirably has a high fluid uptake capability. Fluid uptake is measured in grams of absorbed fluid per gram of absorbent material and is expressed by the value of “maximum uptake.” A high fluid uptake corresponds therefore to a high capacity of the material and is beneficial, because it ensures the complete acquisition of fluids to be absorbed by an acquisition material.

A relevant attribute of the acquisition system 50 is its Median Desorption Pressure, MDP. The MDP is a measure of the capillary pressure that is required to dewater the lower acquisition layer 54 to about 50% of its capacity at 0 cm capillary suction height under an applied mechanical pressure of 0.3 psi. Generally, a relatively lower MDP may be useful. The methods for determining MDP and maximum uptake are disclosed in U.S. patent application Ser. No. 11/600,691 (Flohr et al.).

Suitable non-woven materials for the acquisition system 50 can include, but are not limited to SMS material, comprising a spunbonded, a melt-blown and a further spunbonded layer. In certain embodiments, permanently hydrophilic non-wovens, and in particular, nonwovens with durably hydrophilic coatings are desirable. Another suitable embodiment comprises a SMMS-structure. In certain embodiments, the non-wovens are porous.

In certain embodiments, suitable non-woven materials may include, but are not limited to synthetic fibers, such as PE, PET, and PP. As polymers used for nonwoven production may be inherently hydrophobic, they may be coated with hydrophilic coatings. One way to produce nonwovens with durably hydrophilic coatings, is via applying a hydrophilic monomer and a radical polymerization initiator onto the nonwoven, and conducting a polymerization activated via UV light resulting in monomer chemically bound to the surface of the nonwoven as described in co-pending U.S. Patent Publication No. 2005/0159720. Another way to produce nonwovens with durably hydrophilic coatings is to coat the nonwoven with hydrophilic nanoparticles as described in co-pending applications U.S. Pat. No. 7,112,621 to Rohrbaugh et al. and in PCT Application Publication WO 02/064877.

Typically, nanoparticles have a largest dimension of below 750 nm. Nanoparticles with sizes ranging from 2 to 750 nm may be economically produced. An advantage of nanoparticles is that many of them can be easily dispersed in water solution to enable coating application onto the nonwoven, they typically form transparent coatings, and the coatings applied from water solutions are typically sufficiently durable to exposure to water. Nanoparticles can be organic or inorganic, synthetic or natural. Inorganic nanoparticles generally exist as oxides, silicates, and/or, carbonates. Typical examples of suitable nanoparticles are layered clay minerals (e.g., LAPONITE™ from Southern Clay Products, Inc. (USA), and Boehmite alumina (e.g., Disperal P2™ from North American Sasol. Inc.). According to a certain embodiment, a suitable nanoparticle coated non-woven is that disclosed in the co-pending patent application Ser. No. 10/758,066 entitled “Disposable absorbent article comprising a durable hydrophilic core wrap” to Ekaterina Anatolyevna Ponomarenko and Mattias NMN Schmidt.

Further useful non-wovens are described in U.S. Pat. No. 6,645,569 to Cramer et al., U.S. Pat. No. 6,863,933 to Cramer et al., U.S. Pat. No. 7,112,621 to Rohrbaugh et al., and co-pending patent application Ser. Nos. 10/338,603 to Cramer et al. and 10/338,610 to Cramer et al.

In some cases, the nonwoven surface can be pre-treated with high energy treatment (corona, plasma) prior to application of nanoparticle coatings. High energy pre-treatment typically temporarily increases the surface energy of a low surface energy surface (such as PP) and thus enables better wetting of a nonwoven by the nanoparticle dispersion in water.

Notably, permanently hydrophilic non-wovens are also useful in other parts of an absorbent article. For example, topsheets and absorbent core layers comprising permanently hydrophilic non-wovens as described above have been found to work well.

In certain embodiments, the absorbent core 14 may further comprise any absorbent material that is generally compressible, conformable, non-irritating to the wearer's skin, and capable of absorbing and retaining liquids such as urine and other certain body exudates. In such embodiments, the absorbent core 14 may comprise a wide variety of liquid-absorbent materials commonly used in disposable diapers and other absorbent articles such as comminuted wood pulp, which is generally referred to as airfelt, creped cellulose wadding, melt blown polymers, including co-form, chemically stiffened, modified or cross-linked cellulosic fibers, tissue, including tissue wraps and tissue laminates, absorbent foams, absorbent sponges, or any other known absorbent material or combinations of materials. The absorbent core 14 may further comprise minor amounts (typically less than about 10%) of materials, such as adhesives, waxes, oils and the like.

Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,207 (Dyer et al.); U.S. Pat. No. 5,397,316 (LaVon et al.); and U.S. Pat. No. 5,625,222 (DesMarais et al.).

### C. Methods For Making Absorbent Articles

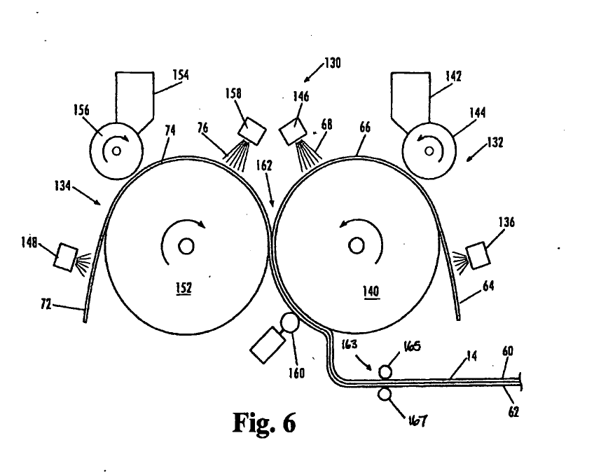

A printing system 130 for making an absorbent core 14 for a diaper 10 in accordance with an embodiment of the invention is illustrated in FIG. 6. The printing system 130 may generally comprise a first printing unit 132 for forming a first absorbent layer 60 of the absorbent core 14 and a second printing unit 134 for forming a second absorbent layer 62 of the absorbent core 14.

The first printing unit 132 may comprise a first auxiliary adhesive applicator 136 for applying an auxiliary adhesive to a substrate 64, which may be a nonwoven web, a first rotatable support roll 140 for receiving the substrate 64, a hopper 142 for holding absorbent particulate polymer material 66, a printing roll 144 for transferring the absorbent particulate polymer material 66 to the substrate 64, and a thermoplastic adhesive material applicator 146 for applying a thermoplastic adhesive material 68 to the substrate 64 and the absorbent particulate polymer 66 material thereon.

The second printing unit 134 may comprise a second auxiliary adhesive applicator 148 for applying an auxiliary adhesive to a second substrate 72, a second rotatable support roll 152 for receiving the second substrate 72, a second hopper 154 for holding an absorbent particulate polymer material 74, a second printing roll 156 for transferring the absorbent particulate polymer material 74 from the hopper 154 to the second substrate 72, and a second thermoplastic adhesive material applicator 158 for applying a thermoplastic adhesive material 76 to the second substrate 72 and the absorbent particulate polymer material 74 thereon.

The printing system 130 also includes a guide roller 160 for guiding the formed absorbent core from a nip 162 between first and second rotatable support rolls 140 and 152.

The first and second auxiliary applicators 136 and 148 and the first and second thermoplastic adhesive material applicators 146 and 158 may be a nozzle system which can provide a relatively thin but wide curtain of thermoplastic adhesive material.

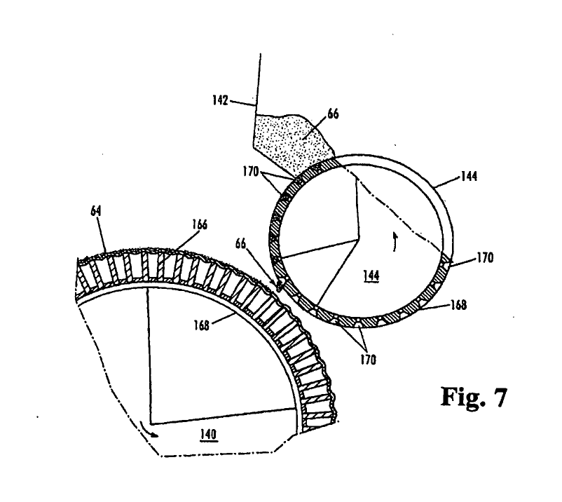

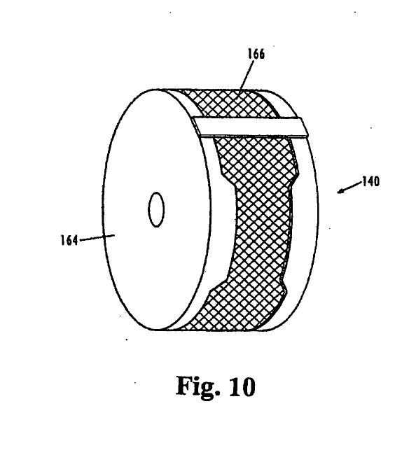

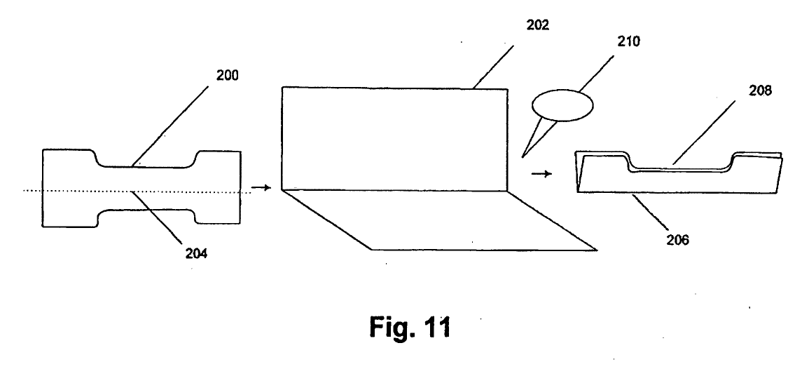

Turning to FIG. 7, portions of the first hopper 142, first support roll 140, and first printing roll 144 are illustrated. As also shown in FIG. 10, the first rotatable support roll 140, which has the same structure as the second rotatable support roll 152, comprises a rotatable drum 164 and a peripheral vented support grid 166 for receiving the first substrate 64.

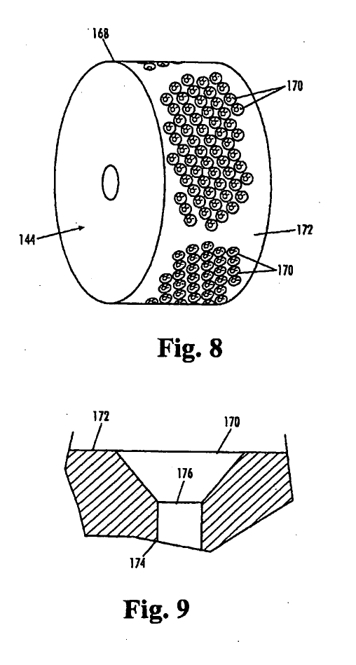

As also illustrated in FIG. 8, the first printing roll 144, which has the same structure as the second printing roll 156, comprises a rotatable drum 168 and a plurality of absorbent particulate polymer material reservoirs 170 in a peripheral surface 172 of the drum 168. The reservoirs 170 best illustrated in FIG. 9, may have a variety of shapes, but in a particular embodiment, are conical. The reservoirs 170 may lead to an air passage 174 in the drum 168 and comprise a vented cover 176 for holding adhesive particulate polymer material 66 in the reservoir and preventing the adhesive particulate polymer material 66 from falling or being pulled into the air passage 174.

A second nip 163 between first and second rotatable compression rolls 165 and 167 can form one or more compression strips, such as 15 in FIG. 1, in the absorbent core 14. In a certain embodiment, the compression rolls 165 and 167 may be a suitable width for forming a similarly sized compression strip in the absorbent core 14. In other certain embodiments, at least one of the compression rolls 165 and 167 may include a suitable pattern for forming a correspondingly sized compression strip in the absorbent core 14. In yet other certain embodiments, one or more jets of pressurized air or another medium could be used to form at least one compression strip in the absorbent core 14.

In operation, the printing system 130 receives the first and second substrate 64 and 72 into the first and second printing units 132 and 134, respectively, the first substrate 64 is drawn by the rotating first support roll 140 past the first auxiliary adhesive applicator 136 which applies the first auxiliary adhesive to the first substrate 64 in a pattern such as described hereinabove. A vacuum (not shown) within the first support roll 140 draws the first substrate 64 against the vertical support grid 166 and holds the first substrate 64 against the first support roll 140. This presents an uneven surface on the first substrate 64. Due to gravity, or by using the vacuum means, the substrate 64 will follow the contours of the uneven surface and thereby the substrate 64 will assume a mountain and valley shape. The absorbent particulate polymer material 66 may accumulate in the valleys presented by the substrate 64. The first support roll 140 then carries the first substrate 64 past the rotating first printing roll 144 which transfers the absorbent particulate polymer material 66 from the first hopper 142 to the first substrate 64 in a grid pattern 92. A vacuum (not shown) in the first printing roll 144 may hold the absorbent particulate polymer material 66 in the reservoirs 170 until time to deliver the absorbent particulate polymer material 66 to the first substrate 64. The vacuum may then be released or air flow through the air passages 174 may be reversed to eject the absorbent particulate polymer material 66 from the reservoirs and onto the first substrate 64. The absorbent particulate polymer material 66 may accumulate in the valleys presented by the substrate 64. The support roll 140 then carries the printed first substrate 64 past the thermoplastic adhesive material applicator 136 which applies the thermoplastic adhesive material 68 to encase the absorbent particulate polymer material 66 on the first substrate 64.

Hence, the uneven surface of the vented support grid 166 of the support rolls 140 and 152 determines the distribution of absorbent particulate polymeric material 66 and 74 throughout the absorbent core 14 and likewise determines the pattern of junction areas.

Meanwhile, the second rotatable support roll draws the second substrate 72 past the second auxiliary adhesive applicator 148 which applies an auxiliary adhesive to the second substrate 72 in any predefined pattern. The second rotatable support roll 152 then carries the second substrate 72 past the second printing roll 156 which transfers the absorbent particulate polymer material 74 from the second hopper 154 to the second substrate 72 and deposits the absorbent particulate polymer material 74 in a grid pattern on the second substrate 72 in the same manner as described with regard to the first printing unit 132 above. The second thermoplastic adhesive material applicator 158 then applies the thermoplastic adhesive material 76 to encase the absorbent particulate polymer material 74 on the second substrate 72. The printed first and second substrates 64 and 72 then pass through the nip 162 between the first and second support rolls 140 and 152 for compressing the first absorbent layer 60 and second absorbent layer 62 together to form the absorbent core 14. The absorbent core 14 then passes through nip 163 between the first and second compression rolls 165 and 167 for forming at least one compression strip in the absorbent core 14. As needed, various lengths of the absorbent core 14 can be cut into predefined sections for use in an absorbent article, such as a diaper 10.

In a further process step, an outer covering 16 may be placed upon or otherwise combined with the substrates 64 and 72, the absorbent particulate polymer material 66 and 74, and the thermoplastic adhesive material 68 and 76. In another embodiment, the outer covering 16 and the respective substrate 64 and 72 may be provided from a unitary sheet of material.

As shown in FIG. 11, after the formed absorbent core 14 is combined with the outer covering 16, the unfinished diaper 200 can be fed to a folding device 202 or process, wherein the unfinished diaper 200 is folded and compressed. In a certain embodiment, the unfinished diaper 200 is folded and compressed along a longitudinal axis 204, similar to 36 in FIG. 1, wherein at least one crease 206 can be formed in the diaper 208. In certain other embodiments, an unfinished diaper 200 can be folded and compressed along one or more lines substantially parallel with the longitudinal axis 204, wherein a corresponding number of creases can be formed in the diaper. When unfolded, the diaper 208 can include a preformed concave V-shape, similar to 58 shown in FIG. 2, within the crotch region of the diaper 208.

For certain embodiments when the absorbent core 14 may be relatively incompressible, such as an airfelt free core, i.e. with little or no wood pulp or cellulosic material, an auxiliary adhesive can be applied to any portion of the crease 206 by an adhesive application device 210. The auxiliary adhesive may be deposited in the crease 206 when the diaper 208 is folded or compressed along the crease 206 by the folding device 202 or process. The auxiliary adhesive may aid in maintaining the relative shape of the crease 206 in the diaper 208.

All patents and patent applications (including any patents which issue thereon) assigned to the Procter & Gamble Company referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a chassis including a topsheet and a backsheet, the chassis having end edges and longitudinal edges;
an absorbent core which comprises an absorbent particulate polymer material, the absorbent core being located between the topsheet and the backsheet, and wherein the absorbent core comprises at least one strip extending through a portion of a crotch region of the article; and
at least one longitudinally oriented crease passing through a portion of the crotch region, wherein a concave shape is formed within the article.

2. The disposable absorbent article of claim 1, wherein the strip is a compressed strip.

3. The disposable absorbent article of claim 1, wherein the absorbent core further comprises at least one cellulosic material.

4. The disposable absorbent article of claim 1, wherein the at least one strip is oriented substantially parallel with a transverse axis associated with the article.

5. The disposable absorbent article of claim 1, wherein the at least one strip extends through a substantial portion of the crotch region.

6. The disposable absorbent article of claim 1, wherein the at least one strip comprises a plurality of strips oriented substantially parallel with a transverse axis associated with the article.

7. The disposable absorbent article of claim 1, wherein the at least one strip comprises at least one of the following: a rectangular-shaped strip, or a polygonal-shaped strip.

8. The disposable absorbent article of claim 1, wherein the at least one longitudinally oriented crease comprises a plurality of longitudinally oriented creases.

9. The disposable absorbent article of claim 1, wherein the at least one longitudinally oriented crease comprises an adhesive.

10. The disposable absorbent article of claim 1, wherein the at least one longitudinally oriented crease extends substantially between the end edges of the article.

11. The disposable absorbent article of claim 1, wherein the concave shape is formed within the crotch region of the article, and wherein the concave shape comprises a shape selected from the group consisting of a concave V-shape, a concave U-shape, and a shape that slopes slightly inward.

12. The disposable absorbent article of claim 1, wherein the absorbent article is a diaper and the concave shape is operable to receive a portion of the wearer's body.

13. A method of manufacturing a disposable absorbent article comprising a chassis including a topsheet and a backsheet, the chassis having end edges and longitudinal edges, and an absorbent core which comprises an absorbent particulate polymer material, the absorbent core being located between the topsheet and the backsheet, the method comprising:
forming at least one strip in a crotch region of the absorbent core;
combining the absorbent core with the chassis; and
compressing the combined absorbent core and chassis, wherein at least one longitudinally oriented crease is formed within the crotch region of the article, and wherein a concave shape is formed within the article.

14. The method of claim 13, wherein the strip is a compressed strip.

15. The method of claim 13, wherein the at least one strip is oriented substantially parallel with a transverse axis associated with the article.

16. The method of claim 13, wherein the at least one strip extends through a substantial portion of the crotch region of the article.

17. The method of claim 13, wherein the at least one strip comprises a plurality of strips oriented substantially parallel with a transverse axis associated with the article.

18. The method of claim 13, wherein the at least one strip comprises at least strip selected from the group consisting of a rectangular-shaped strip and a polygonal-shaped strip; and wherein the concave shape comprises a shape selected from the group consisting of a concave V-shape, a concave U-shape, and a shape that slopes slightly inward.

19. The method of claim 13, wherein the at least one longitudinally oriented crease comprises a plurality of longitudinally oriented creases.

20. The method of claim 13, further comprising applying an adhesive to at least a portion of the at least one longitudinally oriented crease.

21. The method of claim 13, wherein the at least one longitudinally oriented crease extends substantially between the end edges of the article.
